(19) United States
(12) Patent Application Publication
(10) Pub. No.: US 2005/0070606 A1
(43) Pub. Date: Mar. 31, 2005

(54) Pharmaceutical agent for mitochondrial disorders

(75) Inventors: Maiko Mori, Kawasaki-shi (JP); Yoshiyuki Takahara, Kawasaki-shi (JP); Sonoko Ishizaki, Kawasaki-shi (JP); Ichiro Sonaka, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/917,473

(22) Filed: Aug. 13, 2004

Related U.S. Application Data
(63) Continuation of application No. PCT/JP03/01462, filed on Feb. 13, 2003.

(30) Foreign Application Priority Data
Feb. 14, 2002 (JP) 2002-036468

(51) Int. Cl. A61K 31/198

(52) U.S. Cl. 514/561

(57) ABSTRACT

The present invention provides a pharmaceutical agent (e.g., a therapeutic agent) for treatment of mitochondrial disorders, which is highly safe with minimal adverse effects. Thus, the pharmaceutical agent is extremely useful as a medical product for mitochondrial disorders. The present invention also provides a method for preventing, ameliorating and/or therapeutically treating mitochondrial disorders by administering the pharmaceutical agent of the present invention.

### CROSS-REFERENCE TO RELATED APPLICATIONS

The present application is a continuation of International application PCT/JP03/01462, filed on Feb. 13, 2003, and claims priority to Japanese Application No. JP 2002-36468, filed on Feb. 14, 2002, which are hereby incorporated by reference in their entirety.

### BACKGROUND OF THE INVENTION

1. Field of the Invention

The present invention provides a pharmaceutical agent containing alanine, such as L-alanine, for preventing, ameliorating and therapeutically treating mitochondrial disorders (generic name of a series of pathological states occurring due to the functional abnormality of mitochondria). In particular, the present invention provides pharmaceutical agent for mitochondrial disorders, including medical products, such as prophylactic agent, ameliorating agent and therapeutic agent. Further, the invention provides a method for preventing, ameliorating and/or therapeutically treating mitochondrial disorders, by using a pharmaceutical agent containing alanine (e.g, L-alanine).

2. Discussion of the Background

“Mitochondrial disorders” is a generic name for a series of diseases with an etiology of the functional disorder of mitochondria. In general, mitochondria have an energy generating function in cells. It is known that when this function deteriorates, various diseases are induced. Examples of these diseases include MELAS, Leigh's encephalopathy, Huntington disease, Parkinson disease, Alzheimer's disease, MNGIE, chronic progressive external ophthalmoplegia (CPEO), MERRF syndrome, and Leber's disease. It is hypothesized that the diseases are derived from functional abnormalities of various metabolic enzymes existing in mitochondria and it is inferred that the etiology resides in the mutation of the corresponding genes.

Mitochondrial disorders are degenerative diseases due to various mechanisms, including: abnormality of mitochondrial DNA (deletion, point mutation, and duplication), abnormality of cellular DNA encoding mitochondrial enzymes or complex polymeric mitochondrial components, and acquired by toxic substance or pharmaceutical product induction.

When mitochondria-associated genes are damaged due to one or more of these mechanisms, various biochemical abnormalities occur. In other words, camitine palmitoyl transferase deficiency and carnitine deficiency due to substrate transfer disturbances, pyruvate carboxylase deficiency and pyruvate dehydrogenase complex deficiency due to disturbances in substrate use, β-oxidation disturbance, fumarase deficiency and α-ketoglutarate dehydrogenase deficiency due to disturbance in the TCA cycle, Luft disease due to disturbance of oxidative phosphorylation conjugation, complex I deficiency, complex II deficiency, complex III deficiency and complex IV (cytochrome c oxidase) deficiency and complex V (ATP synthase) deficiency due to electron transfer enzyme damages and the like. The essential etiology of these disorders is the mitochondrial energy metabolic disorders. Therefore, the therapeutic methods thereof include therapeutic treatments of the genes as the cause of the disorders, methods for supplementing the enzymes, substrate controls for the damaged enzymes, and making toxic substances non-toxic, or supplementation of lacking substances. Specific therapeutic agents include coenzyme Q₁₀, vitamins, cytochrome formulations, succinic acid, dichloroacetic acid, carnitine, and ATP formulations. Currently, no definite therapeutic agents or therapeutic methods exist.

### SUMMARY OF THE INVENTION

It is an object of the present invention to provide a novel pharmaceutical agent effective for the prophylaxis, amelioration and therapeutic treatment of a series of diseases generically called mitochondrial disorders.

In this object, it is preferred that the pharmaceutical agent contain L-alanine in a form suitable to deliver 10 to 1,000 mg/kg per day (preferably 100 to 500 mg/kg per day) and at least one pharmaceutically acceptable substance. Further, it is preferred that the pharmaceutical agent be in a form suitable for oral consumption.

In other objects of the present invention are

(a) A method for preventing mitochondrial disorders, comprising administering to a subject in need thereof a composition comprising L-alanine in a form suitable to deliver 10 to 1,000 mg/kg per day and at least one pharmaceutically acceptable substance;

(b) A method for preventing mitochondrial disorders, comprising administering to a subject in need thereof a composition comprising L-alanine in a form suitable to deliver 10 to 1,000 mg/kg per day and at least one pharmaceutically acceptable substance; and

(c) A method for preventing mitochondrial disorders, comprising administering to a subject in need thereof a composition comprising L-alanine in a form suitable to deliver 10 to 1,000 mg/kg per day and at least one pharmaceutically acceptable substance.

In these objects, it is preferred that the composition be administered orally and that the subject in need thereof is a human. Moreover, it is preferred that the composition be practiced 2 to 4 times per day. As used herein, the term “mitochondrial disorders” is selected from the group consisting of MELAS, Leigh's encephalopathy, Huntington disease, Parkinson disease, Alzheimer disease, MNGIE, chronic progressive external ophthalmoplegia (CPEO), MERRF syndrome, Leber's disease and diabetes mellitus.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 shows the results of the analysis of gene expression (gene expression of mitochondrial ATP generation-associated protein) in Example 1; where

open square (□) expresses group 2 (control group) and

solid square (▪) expresses group 1 (Ala-dosed group).

FIG. 2 shows the results of the measurement of mitochondrial membrane potential in Example 2 and the influence of L-alanine on mitochondrial membrane potential (JC-1).

FIG. 3 depicts the results of the measurement of COX activity in Example 3 and the influence of L-alanine on the COX activity.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, medical products, food products, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

Because cardiochrome as a complex of cytochrome c with vitamins B₁ and B₂ is prepared from bovine myocardium, potentially, cytochrome c may cause anaphylaxis shock. Additionally, although it has been expected to supplement substrates for cytochrome c oxidase, the effectiveness of the agent is low, when the residual activity of the enzyme is low. Oral cytochrome formulations such as Cytorest are also on the market, however, a sufficient amount of cytochrome may possibly not be delivered in mitochondria when administered orally. Alternatively, it was assumed that alanine might overcome these problems. Thus, the present inventors attempted to administer L-alanine to subjects in need thereof. It was then confirmed that the mRNA expression of cytochrome c oxidase increased and cytochrome c oxidase was activated. Therefore, the effectiveness will possibly be obtained even in a patient with a low residual activity of the enzyme. Additionally, oral alanine administration possibly activates the enzyme, without any risk of anaphylaxis.

Additionally, dichloroacetic acid activates the TCA cycle by enhancing the activity of pyruvate dehydrogenase. When the dose is high, however, side effects such as disordered consciousness occur. In contrast, alanine enhances the mRNA expression of isocitrate dehydrogenase, 2-oxoglutarate carrier, and fumarate hydratase, to activate the TCA cycle with no risk of side effects.

Next, ATP formulations are used for the purpose of supplementing intracellular ATP. Because ATP is readily decomposed, the effect is unknown. In contrast, alanine promotes the activity of ATP synthase by enhancing the mRNA expression of ATP synthase, to increase the ATP amount. Because ATP is generated intracellularly, alanine may possibly be more effective.

Alternatively, succinic acid is the substrate of the complex II. However, it is unknown whether or not succinic acid is sufficiently delivered in mitochondria via its administration route. In contrast, alanine administration increases the mRNA expression of isocitrate dehydrogenase and 2-oxoglutarate carrier in the TCA cycle. This, in turn, can activate isocitrate dehydrogenase in the TCA cycle, to promote succinate generation from 2-oxoglutaric acid. Because succinic acid is generated intracellularly, alanine may possibly be more effective.

Based on the results from the above examination and evaluation, the present inventors found that alanine, such as L-alanine, as one of amino acids activated the TCA cycle and electron transfer system as the important mitochondrial physiological functions and that based on the functions, the pathological state of mitochondrial disorders was prevented, ameliorated and therapeutically treated and the like. Thus, the invention has been achieved on the basis of the finding.

Therefore, the present invention provides a pharmaceutical agent for use in the prophylaxis, amelioration and therapeutic treatment of mitochondrial disorders (referred to as “pharmaceutical agent for mitochondrial disorders”), the pharmaceutical agent containing alanine such as L-alanine as the active ingredient.

The steric configuration of alanine for use in accordance with the invention has not specific limitation. L-form and D-form thereof can be used. For simplicity, L-alanine can be used.

The mitochondrial disorders include for example any pathological states of MELAS, Leigh's encephalopathy, Huntington disease, Parkinson disease, Alzheimer's disease, MNGIE, chronic progressive external ophthalmoplegia (CPEO), MERRF syndrome, Leber's disease and diabetes mellitus.

The dose of the active ingredient alanine of the pharmaceutical agent of the invention is preferably about 10 to 1,000 mg/kg per day, more preferably about 100 to 500 mg/kg per day, still more preferably about 200 to 400 mg/kg per day and can be given into a biological organism requiring the pharmaceutical agent.

The dosage form of the pharmaceutical agent of the invention preferably includes for example but is not limited to tablets, granules, powders, and injections.

In another aspect, the invention relates to a method for preventing, ameliorating and/or therapeutically treating mitochondrial disorders comprising a step of ingesting and administering alanine such as L-alanine to a biological organism. As the form for the ingestion or administration, various forms of the pharmaceutical agent for mitochondrial disorders in accordance with the invention can be selected.

In an additional embodiment, the invention relates to the use of alanine such as L-alanine in producing a pharmaceutical agent for mitochondrial disorders. As to the form of such alanine for use in producing the pharmaceutical agent (the form for use after production), various forms of the pharmaceutical agent for mitochondrial disorders in accordance with the invention can be used.

The present invention provides a pharmaceutical agent using alanine, such as L-alanine, as the active ingredient. The pharmaceutical agent is preferably in a form suitable to be administered into animals (biological organisms), particularly humans, for the prophylaxis, amelioration and therapeutic treatment of mitochondrial disorders.

With the administration of alanine, particularly L-alanine, into biological organisms, the following ameliorating actions for mitochondrial disorders are suggested.

1. With the administration of L-alanine, the mRNA expression of isocitrate dehydrogenase, 2-oxoglutaric acid carrier and fumarate hydratase in the TCA cycle is increased. This, in turn, activates the TCA cycle to promote ATP synthesis. In other words, it ameliorates mitochondrial disorders arising from or manifesting in deficiencies of ATP synthesis, such as MELAS, MERRF syndrome, Leigh's encephalopathy, Parkinson disease, Alzheimer's disease, Huntington disease, Leber's disease, chronic progressive external ophthalmoplegia (CPEO), diabetes mellitus and MNGIE.

2. With the administration of L-alanine, the mRNA expression of isocitrate dehydrogenase and 2-oxoglutaric acid carrier in the TCA cycle is increased. This, in turn, activates the isocitrate dehydrogenase in the TCA cycle to promote succinate generation from 2-oxoglutaric acid. Specifically, the supply of succinic acid as the complex II substrate in the electron transfer system supplements ATP generation from the complex II in mitochondrial disorders with the deficiency of the complex I or the reduction of the activity of the complex I, particularly chronic progressive external ophthalmoplegia (CPEO), Leigh's encephalopathy, Parkinson disease, Alzheimer disease, Huntington disease, Leber's disease and the like, to ameliorate the pathological states thereof.

3. The administration of L-alanine increases the mRNA expression of cytochrome c oxidase in the electron transfer system. Specifically, cytochrome c oxidase is activated. L-Alanine promotes an increase in the activity of cytochrome c oxidase in the Leigh's encephalopathy and Kearns-Sayre syndrome with diffused reduction of cytochrome c oxidase activity, so that the pathological states thereof can be ameliorated.

4. The administration of L-alanine increases the mRNA expression of ATP synthase in the electron transfer system, thus accelerating ATP synthase activity. Consequently, L-alanine accelerates ATP synthesis in diabetes mellitus due to insulin secretion disorders because of the functional deterioration of mitochondria and the reduction of ATP generation, so that insulin secretion is resumed. Additionally, L-alanine accelerates ATP synthesis in mitochondrial disorders with disordered ATP synthesis, such as MELAS, MERRF syndrome, Leigh's encephalopathy, Parkinson disease, Alzheimer's disease, Huntington disease, Leber's disease, chronic progressive external ophthalmoplegia (CPEO) and MNGIE, so that the pathological states thereof can be ameliorated.

In accordance with the invention, a rat after partial 70% hepatectomy as an animal model of liver disorders was used to confirm the efficacy of alanine, particularly L-alanine (see the following Examples). The animal model was reported to have reduced mitochondrial functions.

The dosage and dosing form of the pharmaceutical agent of the invention are not specifically limited. Therefore, various dosing forms can be adopted, including for example oral administration and parenteral administration (intravenous administration and the like, intraperitoneal administration, transdermal administration, inhalation administration, infusion administration and the like). For example, tablets, granules, powders, and injections can be adopted as the dosing forms.

The dose of the pharmaceutical agent of the invention can appropriately be selected, depending on the type (pathological states) of a mitochondrial disorder and the severity of the symptoms, and the form (dosing form) of the formulation. In case that alanine, for example L-alanine, is to be administered orally, a dose of L-alanine is preferably about 10 to 1000 mg/kg, more preferably about 100 to 500 mg/kg, and most preferably about 200 to 400 mg/kg per day per such patient. In case of a severe case, the dose can be increased further. Concerning the number and timing of dosing, the dose can be administered once in several days or once daily. Generally, the dose can be divided in several portions, for example 2 to 4 portions for sustained administration.

In the case of parenteral dosing, the dose can be selected and administered on the basis of a dose of about 1/10- to 1/20-fold the dose for oral dosing as described above.

The dose calculation is carried out in a manner corresponding to the range of the dose of alanine, such as L-alanine, contained in the pharmaceutical agent of the invention. The amount of alanine, particularly L-alanine derived from food and drink products, nutrition, and other medical products as supplied through a route different from the administration route of the pharmaceutical agent to animals, particularly patients to be administered with the pharmaceutical agent is never included in the calculation of the dose. Therefore, a patient with a mitochondrial disorder or the like to which the pharmaceutical agent is to be administered can reasonably ingest L-alanine separately from the object of the present invention, for example in the daily dietary life.

Additionally, the formulation may contain various pharmacologically acceptable substances, including auxiliary agents and the like. A pharmacologically acceptable substance for formulation can be selected depending on the dosage form of the formulation. For example, the pharmacologically acceptable substances may include excipients, diluents, additives, disintegrators, binders, coating agents, lubricants, sliding agents, lubricating agent, flavors, sweeteners and solubilizers, and mixtures thereof. Further, the pharmacologically acceptable substances may be one or more of the following specific substances: magnesium carbonate, titanium dioxide, lactose, mannitol and other sugars, talc, milk protein, gelatin, starch, cellulose and derivatives thereof, animal and vegetable oils, polyethylene glycol, and solvents, for example sterilized water and monohydric and polyhydric alcohols, for example glycerol.

Forms of these various medical formulations include, for example, appropriate solid or liquid formulations, for example granules, powders, coated tablets, tablets, (micro) capsules, suppositories, syrup, juice, suspensions, emulsions, drops, injection solutions and formulations for sustained release of the active substance.

The pharmaceutical agent of the invention in the form of the formulation listed above should reasonably contain the ingredient alanine at an amount effective for the exertion of the pharmaceutical efficacy. With reference to the dose and the like, the active ingredient can be blended in the pharmaceutical agent.

In another aspect as described above, the invention relates to a method for preventing, ameliorating and/or therapeutically treating mitochondrial disorders, including a step of allowing biological organisms to ingest alanine such as L-alanine or administering alanine such as L-alanine to biological organisms. In an additional aspect, the invention relates to the use of alanine such as L-alanine in producing a pharmaceutical agent for mitochondrial disorders.

All these inventions, particularly including the ingestion or administration form or the form for the use can be carried out readily with reference to the descriptions about the pharmaceutical agent for mitochondrial disorders in accordance with the invention and descriptions about the following Examples and the like and additionally with reference to the related art if necessary.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

### Expression of the Genes for Mitochondrial ATP Generation-Associated Proteins

Male F344/DvCrj (Fischer) rats (age 6 weeks and body weight of 120 g) were preliminarily fed with CRF-1 (Oriental Yeast) and water for 6 weeks. Subsequently, the rats were divided in two groups of equal body weight. According to the Higgins-Anderson method (see Higgins, G. M. and Anderson, R. M., 1931, Arch. Pathol. 12: 186-191), partial 70% hepatectomy (left lobe, intermediate lobe) was performed. 18 and 21 hours after the partial hepatectomy, L-alanine at 2 g/10 ml/kg body weight in aqueous 0.3% carboxymethyl cellulose solution was administered orally (group 1), while only the aqueous 0.3% carboxymethyl cellulose solution was administered to the control group (group 2). 24 hours after the partial hepatectomy, anatomy was performed. The rats were killed by exsanguination under anesthesia and then the liver was resected and weighed. Subsequently, the liver was frozen and stored for assaying mRNA expression by the Taqman-PCR method.

To homogenize the liver, 10 ml ISOGEN (Nippon Gene) was added per 1 g of rat liver. The resulting homogenate was centrifuged to recover the supernatant. Chloroform (200 μl per 1 ml of ISOGEN) was added to the supernatant and gently agitated. After the resulting mixture was incubated at ambient temperature for 2 minutes, the mixture was centrifuged at 15,000 rpm and 4° C. for 10 minutes to recover the aqueous layer. An equal volume of 2-propanol was added to the aqueous layer and the resulting mixture was incubated at ambient temperature for 5 minutes and centrifuged at 15,000 rpm and 4° C. for 15 minutes. The supernatant was then discarded and the precipitated pellet was resuspended in 70% ethanol. The resulting mixture was centrifuged at 15,000 rpm and 4° C. for 15 minutes and the pellet was recovered. The pellet was dried at ambient temperature for 5 minutes, to which DEPC (diethylpyrocarbonate)-treated water was added to solubilize the pellet.

The Taqman PCR template cDNA was synthesized using SuperScript First-Strand Synthesis System for RT-PCR (manufactured by GIBCO BRL). 500 ng of total RNA, 1 μl of 0.5 μg/μl Oligo (dT)₁₂₋₁₈, and 1 μl of 10 mM dNTP mix were dissolved in the DEPC-treated water at a final volume of 10 μl. After reacting the mixture at 65° C. for 5 minutes, the reaction mixture was cooled with ice, followed by addition of 2 μl of 10×RT buffer, 4 μl of 25 mM MgCl₂, 2 μl of 0.1 M DTT, and 1 μl of RNase Inhibitor. The resulting mixture was mixed together and then incubated at 42° C. for 2 minutes. Subsequently, 1 μl (50 units) of a reverse transcriptase SUPERSCRIPT II RT was added and the resultant mixture was reacted together at 42° C. for 50 minutes followed by 70° C. for 15 minutes.

Primers for the proteins involved in the mitochondrial TCA cycle and the ATP generation in the electron transfer system were designed. For the designing, an outside database Primer 3 was used. (http://www.genome.wi.mit.edu/cgi-bin/primer/primer3_www.cgi)

Table 1 shows the types of the genes and the Unigene Nos. (http://www.ncbi.nlm.nih.gov/Unigene), and the nucleotide sequences of the primers (see SEQ ID Nos. 1-26 in the Sequence listing).

TABLE 1

| | |
|---|---|
| Unigene | Primer sequence |

| | | | |
|---|---|---|---|
| NO.(Rn.) | Name of gene | 5′ terminus | 3′ terminus |

| | | | |
|---|---|---|---|
| 1093 | Rattus | tca gct tcc aac atg cta cg | ctt gcc aac ctt gat cac ct |
| | norvegicus mRNA | (SEQ ID NO: 1) | (SEQ ID NO: 2) |
| | for NAD+ | | |
| | specific | | |
| | isocitrate | | |
| | dehydrogenase | | |
| | b-subunit, | | |
| | partial cds | | |
| 2837 | NAD(H)-specific | tgg gtg atg gac tct tcc tc | gct gca ttg ttg tgt tgt cc |
| | isocitrate | (SEQ ID NO: 3) | (SEQ ID NO: 4) |
| | dehydrogenase | | |
| | gamma subunit | | |
| 29782 | Fumarate | aag aa | at |
| | hydratase | (SEQ ID NO: 5) | (SEQ ID NO: 6) |
| 880 | Cytochrome c | tgg acc ctg act ctt gtg tg | aag gga tgg agg agc aaa gt |
| | oxidase subunit | (SEQ ID NO: 7) | (SEQ ID NO: 8) |
| | VIa (liver) | | |
| 1745 | Cytochrome c | gtt cag tag tcg cgg ttg gt | gaa gtg gtg ctg atg gtc ct |
| | oxidase subunit | (SEQ ID NO: 9) | (SEQ ID NO: 10) |
| | VIIa 3 | | |
| 2270 | Rattus norvegicus | ttc ctg ctt cgt gtg ttg tc | tca aag gat gag gga aga cg |
| | liver cytochrome | (SEQ ID NO: 11) | (SEQ ID NO: 12) |
| | c oxidase | | |
| | subunit VIII | | |
| | (COX-VIII) | | |
| | mRNA, 3′ end | | |
| | of cds | | |
| 19207 | Rattus norvegicus | tgt gcg atc gtt act gct tt | agg gct tca gag gct tct tc |
| | mRNA for cytochrome | (SEQ ID NO: 13) | (SEQ ID NO: 14) |
| | C oxidase assembly | | |
| | protein COX17, | | |
| | complete cds | | |
| 10249 | Cytochrome b5, | cca tcg tgg gtg cta ttc tt | agg aga tgt gct ccg aca ct |
| | outer mitochon- | (SEQ ID NO: 15) | (SEQ ID NO: 16) |
| | drial membrane | | |
| | isoform | | |
| 80 | ATP synthase | aac gct ctg aag tcc tgg aa | gtc cac att ggc cct gta gt |
| | subunit d | (SEQ ID NO: 17) | (SEQ ID NO: 18) |
| 3357 | ATP synthase, | aaa aat gca gac cac gaa gg | cta ctc agg agg gag gca ga |
| | H+ trans- | (SEQ ID NO: 19) | (SEQ ID NO: 20) |
| | porting, mito- | | |
| | chondrial F0 | | |
| | complex, subunit | | |
| | c (subunit 9), | | |
| | isoform 1 | | |
| 9723 | Rattus norvegicus | gct gac gtg ctg cag aat ta | atc ctg gca ctc tgc tca ct |
| | (clone gamma-3) | (SEQ ID NO: 21) | (SEQ ID NO: 22) |
| | ATP synthase | | |
| | gamma-subunit | | |
| | (ATP5c) mRNA, | | |
| | 3′ end cds | | |
| 853 | 2-oxoglutarate | ttt ctt cag cct gtg gaa gg | gac gct tgt agg cct tgt tc |
| | | (SEQ ID NO: 23) | (SEQ ID NO: 24) |
| 69 | β-actin | aaa tgc ttc tag gcg gac tg | aaa gcc atg cca aat gtc tc |
| | | (SEQ ID NO: 25) | (SEQ ID NO: 26) |

The reaction mixture of the composition shown below in Table 2 was mixed in a PCR tube for Taqman, and the PCR reaction was performed with an ABI7700 Prism Sequence Detector. The reaction conditions were as follows.

A cycle composed of 50° C. for 2 minutes→95° C. for 10 minutes→(95° C. for 15 seconds→60° C. for 1 minute) was carried out 40 times.

TABLE 2

| Composition of PCR reaction mixture | (per tube) | |
|---|---|---|
| Template cDNA (corresponding to 2.5 ng of total RNA) | 0.1 | μl |
| 5 units/μl AmpliTaq Gold | 0.05 | μl |
| DNTP mix (each 2.5 mM) | 0.8 | μl |
| 25 mM MgCl₂ | 1.2 | μl |
| 10 × SYBR buffer | 1 | μl |
| Primer solution (5′ terminus) | 1 | μl |
| Primer solution (3′ terminus) | 1 | μl |
| DH₂O | total: 10 | μl |

The Taqman PCR reactions were performed in duplicate to calculate the cycle time (CT) exceeding the threshold. The expression level at CT=30 was defined as 1. By the following formula, the relative expression level was determined:
Relative expression level=2^{(30-CT sample)}

Further, the relative value of the expression level of each gene was determined when the relative expression level of β-actin was defined as 1000.

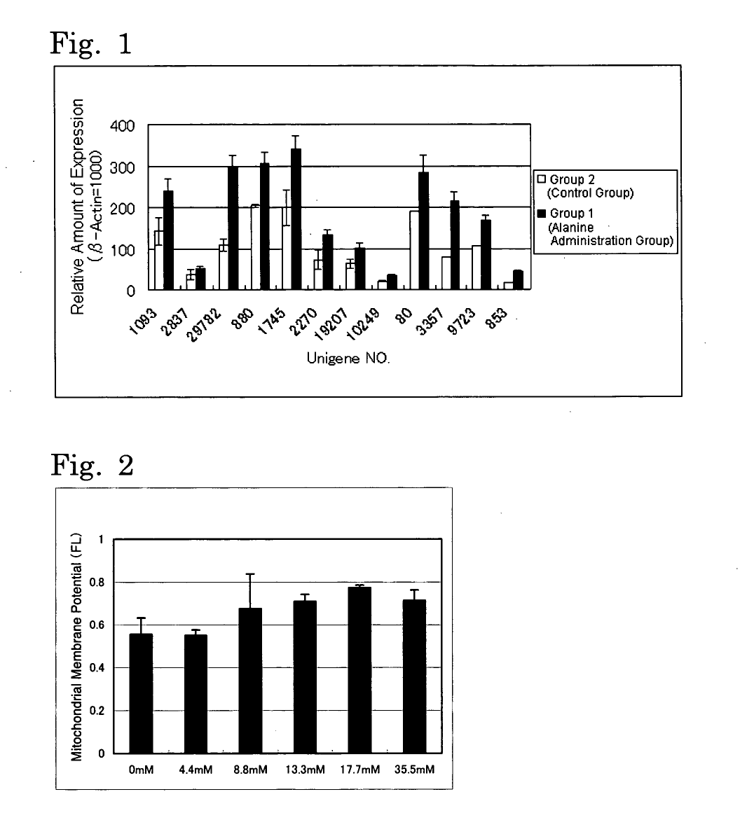

The results of the analysis of gene expression are shown in FIG. 1. As demonstrated by FIG. 1, administration of L-alanine significantly increased the mRNAs of the proteins involved in the TCA cycle of liver mitochondria and the ATP generation in the electron transfer system, compared with the non-administered group. Thus, L-alanine improved the energy generation in liver mitochondria.

### Example 2

### Assaying Mitochondrial Membrane Potential

A male SD (IGS) rat of age 8 weeks was killed by exsanguination under ether anesthesia and then the liver was resected and washed twice with 1×Extraction Buffer A (10 mM HEPES (2-[4-(2-hydroxyethyl)-1-piperazinyl]ethanesulfonic acid, pH 7.5, 200 mM mannitol, 70 mM sucrose, 1 mM EGTA (ethylene glycol bis(β-aminoethyl ether)-N, N,N,N-tetraacetic acid). The liver was subsequently cut into a piece of about 100 mg and weighed, which was then cut into smaller pieces in a 5-ml tube.

A 10-fold volume of 1×Extraction Buffer A (containing 2 mg/ml albumin) was added to the tube and the liver was homogenized at a low speed on ice (15 seconds). The resulting homogenate was transferred into an Eppendorf tube for centrifugation at 600 g at 4° C. for 5 minutes. The supernatant was transferred to a new tube for centrifugation at 11,000 g and 4° C. for 10 minutes. After the supernatant was discarded, the precipitate was suspended in a 10-fold volume of 1×Extraction Buffer A. The procedure from centrifugation at 600 g and 4° C. for 5 minutes to centrifugation at 11,000 g and 4° C. for 10 minutes was repeated. The resulting supernatant was discarded and the precipitate was suspended in 40 μl of a storage buffer (10 mM HEPES pH 7.4, 250 mM sucrose, 1 mM ATP, 0.08 mM ADP (adenosine-5′-diphosphate), 5 mM sodium succinate, 2 mM K₂HPO₄, 1 mM DTT).

The resulting suspension was defined as the mitochondrial suspension for the following experiments. Through protein assay, the mitochondrial suspension was adjusted to 1 mg/ml. 2 μl (2 μg) of the mitochondrial suspension was mixed with 190 μl of an assay buffer (20 mM MOPS (3-(N-morpholino)propanesulfonic acid), pH 7.5, 100 mM KCl, 10 mM ATP, 10 mM MgCl₂, 10 mM sodium succinate, 1 mM EGTA), to which various concentrations of L-alanine were added. For assaying fluorescence intensity (excitation at 490 nm; emission at 590 nm), 2 μl of JC-1 (1 μg/ml) was added and incubated at ambient temperature for 7 minutes. The results are shown in FIG. 2. Consequently, it was shown that the fluorescence intensity of JC-1 was increased in an L-alanine concentration-dependent manner. This suggests that the electron transfer system of mitochondria is activated to increase the membrane potential.

The results described above indicate that L-alanine is promising as an active ingredient for pharmaceutical agents for mitochondrial disorders.

### Example 3

### Assaying COX (cytochrome C oxidase) Activity

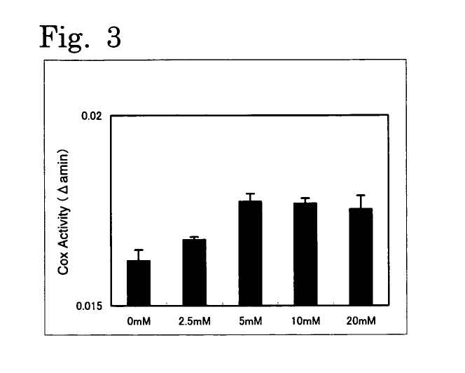

As described in Example 2, a mitochondrial suspension was prepared and adjusted to 0.1 mg/ml. 25 μl (2.5 μg) of mitochondria was blended with 950 μl of an assay buffer (10 mM Tris-HCl, pH 7.0, 120 mM KCl), to which various concentrations of L-alanine were added. 50 μl of cytochrome c (0.22 mM) reduced with 10 mg/ml sodium hydroxysulfite was added, and the resulting mixture was inversed and mixed together to start reaction. Immediately, the absorbance (550 nm) was counted continuously for one minute with a spectrophotometer, to record the decrease of the absorbance. The results are shown in FIG. 3, which evidence that L-alanine increased the COX activity in a concentration-dependent manner from 2.5 mM. It was suggested that the activation of the COX activity was partially involved in the activation of the mitochondrial membrane with L-alanine in Example 2.

The results described above show that alanine, particularly L-alanine, can be used as an active ingredient for pharmaceutical agents for mitochondrial disorders.

Numerous modifications and variations on the present invention are possible in light of the above teachings. It is, therefore, to be understood that within the scope of the accompanying claims, the invention may be practiced otherwise than as specifically described herein.

1. A pharmaceutical agent for mitochondrial disorders, consisting essentially of L-alanine in a form suitable to deliver 10 to 1,000 mg/kg per day and at least one pharmaceutically acceptable substance.

2. The pharmaceutical agent of claim 1, which is in a form suitable for oral consumption.

3. A method for preventing mitochondrial disorders, comprising administering to a subject in need thereof a composition comprising L-alanine in a form suitable to deliver 10 to 1,000 mg/kg per day and at least one pharmaceutically acceptable substance.

4. The method of claim 3, wherein said composition is administered orally.

5. The method of claim 3, wherein said subject is a human.

6. The method of claim 3, wherein said composition comprising L-alanine is in a form suitable to deliver 100 to 500 mg/kg per day.

7. The method of claim 3, wherein said composition is administered to said subject daily.

8. The method of claim 3, wherein said composition is administered to said subject 2 to 4-times daily.

9. The method of claim 3, wherein said mitochondrial disorders is selected from the group consisting of MELAS, Leigh's encephalopathy, Huntington disease, Parkinson disease, Alzheimer disease, MNGIE, chronic progressive external ophthalmoplegia (CPEO), MERRF syndrome, Leber's disease and diabetes mellitus.

10. A method for ameliorating mitochondrial disorders, comprising administering to a subject in need thereof a composition comprising L-alanine in a form suitable to deliver 10 to 1,000 mg/kg per day and at least one pharmaceutically acceptable substance.

11. The method of claim 10, wherein said composition is administered orally.

12. The method of claim 10, wherein said subject is a human.

13. The method of claim 10, wherein said composition comprising L-alanine is in a form suitable to deliver 100 to 500 mg/kg per day.

14. The method of claim 10, wherein said composition is administered to said subject daily.

15. The method of claim 10, wherein said composition is administered to said subject 2 to 4-times daily.

16. The method of claim 10, wherein said mitochondrial disorders is selected from the group consisting of MELAS, Leigh's encephalopathy, Huntington disease, Parkinson disease, Alzheimer disease, MNGIE, chronic progressive external ophthalmoplegia (CPEO), MERRF syndrome, Leber's disease and diabetes mellitus.

17. A method for treating mitochondrial disorders, comprising administering to a subject in need thereof a composition comprising L-alanine in a form suitable to deliver 10 to 1,000 mg/kg per day and at least one pharmaceutically acceptable substance.

18. The method of claim 17, wherein said composition is administered orally.

19. The method of claim 17, wherein said subject is a human.

20. The method of claim 17, wherein said composition comprising L-alanine is in a form suitable to deliver 100 to 500 mg/kg per day.

21. The method of claim 17, wherein said composition is administered to said subject daily.

22. The method of claim 17, wherein said composition is administered to said subject 2 to 4-times daily.

23. The method of claim 17 wherein said mitochondrial disorders is selected from the group consisting of MELAS, Leigh's encephalopathy, Huntington disease, Parkinson disease, Alzheimer disease, MNGIE, chronic progressive external ophthalmoplegia (CPEO), MERRF syndrome, Leber's disease and diabetes mellitus.


SEQUENCE LISTING (26 sequences)

SEQ ID NO: 1 - DNA, 20 bp, Artificial Sequence
  Synthetic DNA
tcagcttccaacatgctacg

SEQ ID NO: 2 - DNA, 20 bp, Artificial Sequence
  Synthetic DNA
cttgccaaccttgatcacct

SEQ ID NO: 3 - DNA, 20 bp, Artificial Sequence
  Synthetic DNA
tgggtgatggactcttcctc

SEQ ID NO: 4 - DNA, 20 bp, Artificial Sequence
  Synthetic DNA
gctgcattgttgtgttgtcc

SEQ ID NO: 5 - DNA, 20 bp, Artificial Sequence
  Synthetic DNA
gccaagactgcacacaagaa

SEQ ID NO: 6 - DNA, 20 bp, Artificial Sequence
  Synthetic DNA
cttgggtttcacccactcat

SEQ ID NO: 7 - DNA, 20 bp, Artificial Sequence
  Synthetic DNA
tggaccctgactcttgtgtg

SEQ ID NO: 8 - DNA, 20 bp, Artificial Sequence
  Synthetic DNA
aagggatggaggagcaaagt

SEQ ID NO: 9 - DNA, 20 bp, Artificial Sequence
  Synthetic DNA
gttcagtagtcgcggttggt

SEQ ID NO: 10 - DNA, 20 bp, Artificial Sequence
  Synthetic DNA
gaagtggtgctgatggtcct

SEQ ID NO: 11 - DNA, 20 bp, Artificial Sequence
  Synthetic DNA
ttcctgcttcgtgtgttgtc

SEQ ID NO: 12 - DNA, 20 bp, Artificial Sequence
  Synthetic DNA
tcaaaggatgagggaagacg

SEQ ID NO: 13 - DNA, 20 bp, Artificial Sequence
  Synthetic DNA
tgtgcgatcgttactgcttt

SEQ ID NO: 14 - DNA, 20 bp, Artificial Sequence
  Synthetic DNA
agggcttcagaggcttcttc

SEQ ID NO: 15 - DNA, 20 bp, Artificial Sequence
  Synthetic DNA
ccatcgtgggtgctattctt

SEQ ID NO: 16 - DNA, 20 bp, Artificial Sequence
  Synthetic DNA
aggagatgtgctccgacact

SEQ ID NO: 17 - DNA, 20 bp, Artificial Sequence
  Synthetic DNA
aacgctctgaagtcctggaa

SEQ ID NO: 18 - DNA, 20 bp, Artificial Sequence
  Synthetic DNA
gtccacattggccctgtagt

SEQ ID NO: 19 - DNA, 20 bp, Artificial Sequence
  Synthetic DNA
aaaaatgcagaccacgaagg

SEQ ID NO: 20 - DNA, 20 bp, Artificial Sequence
  Synthetic DNA
ctactcaggagggaggcaga

SEQ ID NO: 21 - DNA, 20 bp, Artificial Sequence
  Synthetic DNA
gctgacgtgctgcagaatta

SEQ ID NO: 22 - DNA, 20 bp, Artificial Sequence
  Synthetic DNA
atcctggcactctgctcact

SEQ ID NO: 23 - DNA, 20 bp, Artificial Sequence
  Synthetic DNA
tttcttcagcctgtggaagg

SEQ ID NO: 24 - DNA, 20 bp, Artificial Sequence
  Synthetic DNA
gacgcttgtaggccttgttc

SEQ ID NO: 25 - DNA, 20 bp, Artificial Sequence
  Synthetic DNA
aaatgcttctaggcggactg

SEQ ID NO: 26 - DNA, 20 bp, Artificial Sequence
  Synthetic DNA
aaagccatgccaaatgtctc